(19) United States
(12) Patent Application Publication
(10) Pub. No.: US 2012/0323185 A1
(43) Pub. Date: Dec. 20, 2012

(54) Automatic retractable safety syringe

(75) Inventor: Feng Chih Ho, Taipei (TW)

(73) Assignee: Oxti Corporation

(21) Appl. No.: 13/134,780

(22) Filed: Jun. 15, 2011

(51) Int. Cl. A61M 5/32

(52) U.S. Cl. 604/192

(57) ABSTRACT

A syringe includes a barrel having a mouth on one end portion, a needle seat and a needle engaged in the mouth of the barrel, a plunger slidably engaged into the barrel, a packing releasably engaged in the plunger for forming a vacuum within the plunger, and an engaging member attached to the packing and extended out of the plunger for engaging with and for moving the needle seat and the needle into the plunger, and the packing and the engaging member and the needle are forced to move into the plunger by the vacuum of the plunger when the packing is forced to move relative to the plunger and when a releasable engagement between the plunger and the packing is spoiled.

### BACKGROUND OF THE INVENTION

1. Field of the Invention

The present invention relates to an automatic retractable safety syringe, and more particularly to an automatic retractable safety syringe including a simplified structure that may be easily and quickly manufactured and assembled and that may include a greatly reduced or decreased manufacturing cost and that may be easily actuated or operated by the user.

2. Description of the Prior Art

Typical syringes comprise a piston slidably received or engaged in an outer cylindrical member or barrel, and a piston rod attached or mounted or secured or coupled to the piston for moving the piston along or relative to the cylindrical member or barrel, and for selectively drawing the fluid or medicine into and out of the cylindrical member or barrel.

Due to the recent advent of the AIDS virus, which may be contracted by contaminated hypodermic syringes, there have been several retractable needle hypodermic syringes invented and patented.

For example, U.S. Pat. No. 5,785,687 to Saito, U.S. Pat. No. 6,093,171 to Huang, U.S. Pat. No. 6,196,997 to Saito, and U.S. Pat. No. 6,482,174 to Tiu disclose several of the typical retractable safety syringes each comprising a hollow plunger slidably received or engaged in an outer cylindrical member or barrel, and a piston slidably received or engaged in the plunger for retracting the needle into the plunger after use.

However, in the typical retractable safety syringes, the users have to draw the needle into the plunger after use by themselves, and the needle may not be drawn into the plunger automatically without being operated by the user.

U.S. Pat. No. 7,476,215 to Lam discloses one of the typical automatic retractable safety syringes comprising a hollow plunger slidably received or engaged in an outer cylindrical member or barrel, and a piston slidably received or engaged in the plunger, and a piston vacuum seal required to be attached or mounted or secured to the distal end of the piston, the relative axial movement of the plunger in relation to the piston causes a vacuum to be created within the plunger.

However, the typical automatic retractable safety syringes comprises a number of parts or elements that are required to be precisely assembled. In addition, the piston vacuum seal that is attached or mounted or secured to the distal end of the piston has a good chance to be tilted or inclined relative to the plunger, and the vacuum created within the plunger may have a good chance to become failure.

The present invention has arisen to mitigate and/or obviate the afore-described disadvantages of the conventional automatic retractable safety syringes.

### SUMMARY OF THE INVENTION

The primary objective of the present invention is to provide an automatic retractable safety syringe including a simplified structure that may be easily and quickly manufactured and assembled and that may include a greatly reduced or decreased manufacturing cost and that may be easily actuated or operated by the user.

In accordance with one aspect of the invention, there is provided a syringe comprising a barrel including a chamber formed therein, and including an opening formed in a first end portion thereof, and including a mouth formed in a second end portion thereof, a needle seat engaged in the mouth of the barrel and including a needle attached to the needle seat, a plunger slidably engaged into the opening and the chamber of the barrel, and including a first end portion located out of the barrel, and including a compartment formed therein, and including a second end portion, and a packing releasably engaged into the compartment at the second end portion of the plunger for forming a vacuum within the plunger, and an engaging member attached to the packing and extended out of the plunger for selectively engaging with the needle seat and for selectively moving the needle seat and the needle into the compartment of the plunger, and the packing and the engaging member are forced to move relative to the plunger and to spoil a releasable engagement between the plunger and the packing when the plunger is forced and moved toward the needle seat, and the packing and the engaging member and the needle seat and the needle are automatically drawn into the compartment of the plunger by the vacuum of the plunger when the packing is forced to move relative to the plunger.

The barrel includes a gasket engaged into the chamber of the barrel and located close to the mouth of the barrel for engaging with the second end portion of the plunger and for selectively applying a reaction force onto the packing and the engaging member when the plunger is forced and moved toward the needle seat.

The plunger includes an insert engaged into the compartment at the first end portion of the plunger for forming the vacuum within the plunger and between the insert and the packing. The insert is secured to the barrel with a fastener or lock or latch device. The insert includes a sealing ring engaged with the plunger for making a fluid tight seal between the insert and the plunger.

The plunger includes a sealing ring disposed on the second end portion of the plunger and engaged with the barrel or engaged between the plunger and the barrel for making a fluid tight seal between the barrel and the plunger.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a cross sectional view of an automatic retractable safety syringe in accordance with the present invention;

FIG. 2 is an enlarged partial cross sectional view of the automatic retractable safety;

FIGS. 3, 4 are cross sectional views similar to FIG. 1, illustrating the operation of the automatic retractable safety syringe;

FIGS. 5, 6 are enlarged partial cross sectional views illustrating the operation of the automatic retractable safety syringe; and

FIG. 7 is a further cross sectional view illustrating the operation of the automatic retractable safety syringe.

### DETAILED DESCRIPTION OF THE PREFERRED EMBODIMENT

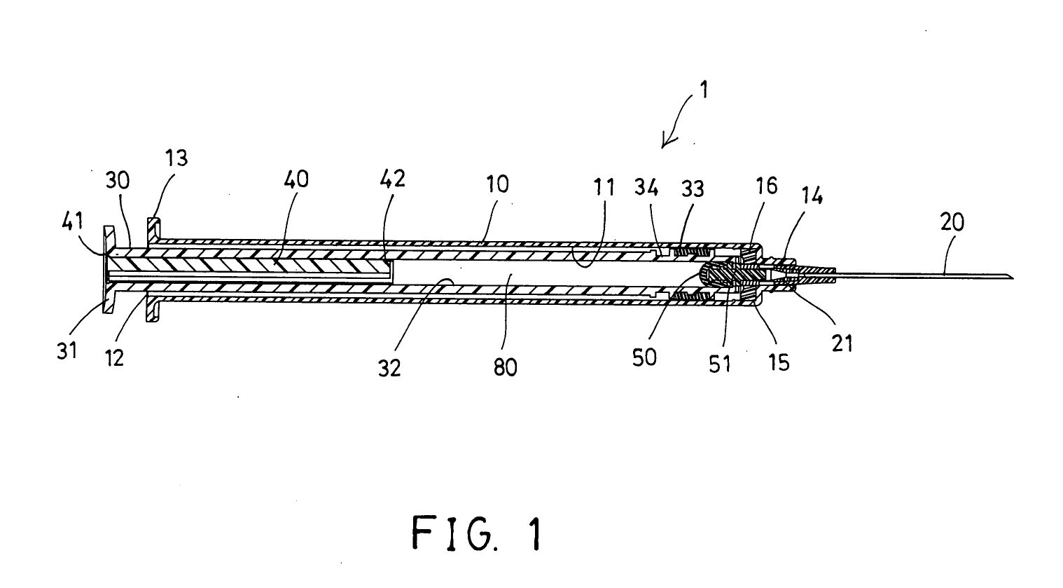

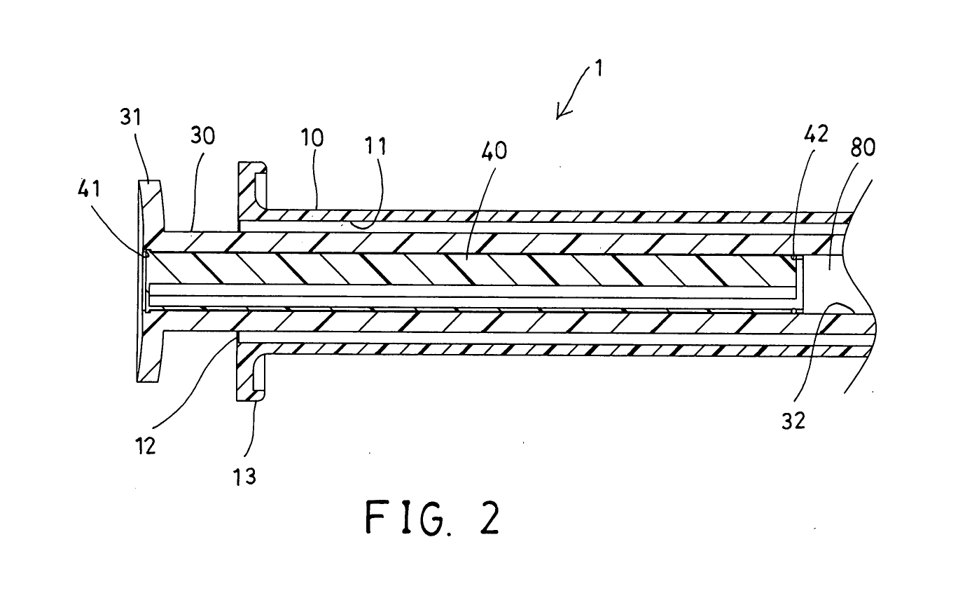

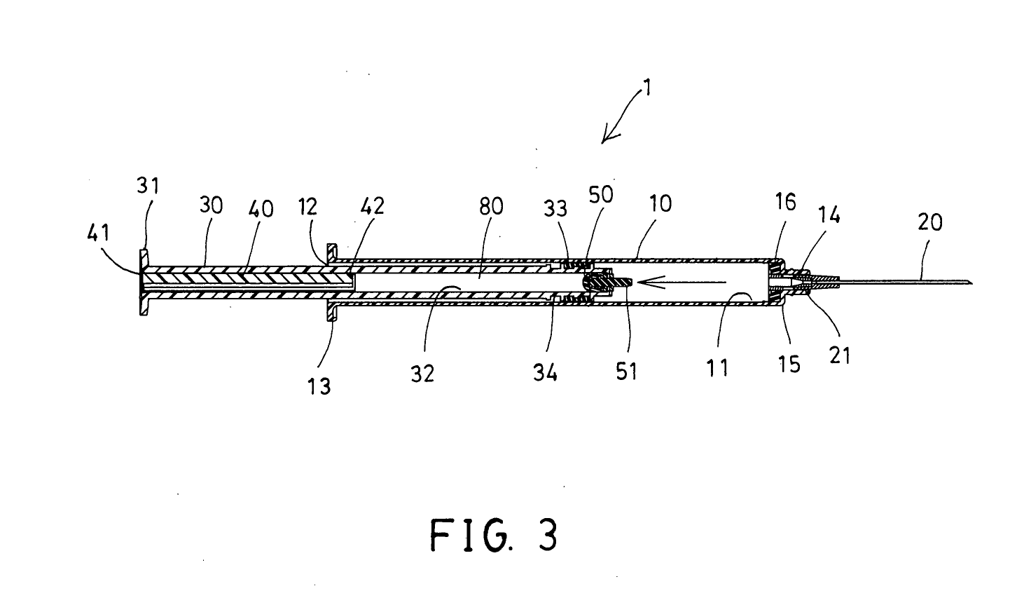

Referring to the drawings, and initially to FIGS. 1-6, an automatic retractable safety syringe 1 in accordance with the present invention comprises a barrel 10 including a longitudinal chamber 11 formed therein, and including an opening 12 formed or provided in the first or one end portion 13 thereof for slidably receiving or engaging with a plunger 30, and including a mouth 14 formed or provided in the second or other end portion 15 thereof, and including a sealing ring or gasket 16 disposed or engaged into the chamber 11 of the barrel 10 and contacted or located close to the mouth 14 of the barrel 10, best shown in FIGS. 3 and 5-7.

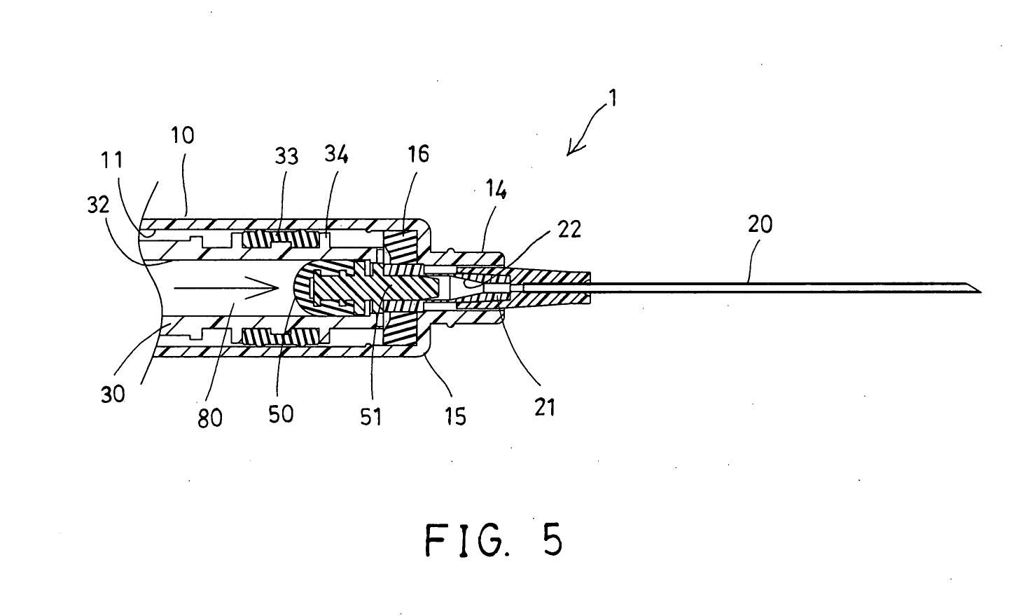

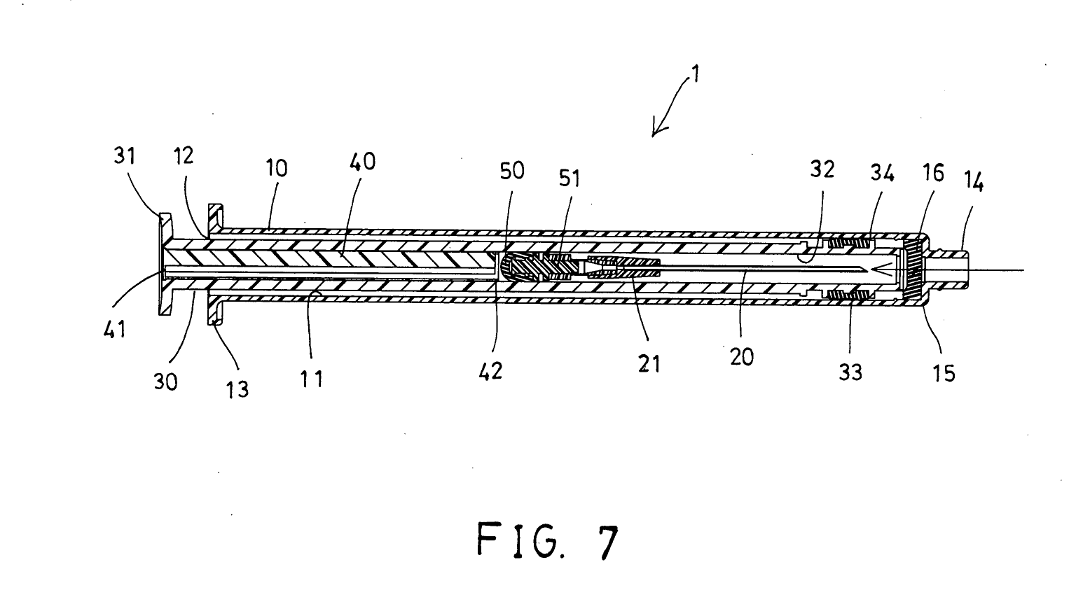

A needle 20 is attached or mounted or secured to a needle seat 21, or includes a needle seat 21 attached or mounted thereto and releasably disposed or engaged into the mouth 14 of the barrel 10, and movable or engageable into the chamber 11 of the barrel 10 (FIG. 7), and the needle seat 21 preferably includes a cone-shaped or frustum-shaped or tilted or inclined inner peripheral surface 22 formed therein (FIG. 5).

The plunger 30 is slidably received or engaged in the chamber 11 of the barrel 10, and includes a first or one end portion 31 extended or located out of the barrel 10, and includes a bore or longitudinal compartment 32 formed therein, and includes a gasket or sealing ring 33 disposed or engaged onto the outer peripheral portion of the second or other end portion 34 of the plunger 30 and contacted or engaged with the barrel 10 for making an air or water or fluid tight seal between the barrel 10 and the plunger 30.

A filling or sealing member or insert 40 is disposed or engaged into the compartment 32 at the one end portion 31 of the plunger 30 and attached or mounted or secured or anchored or retained to the barrel 10 with a fastener or lock or latch device 41, and includes a gasket or sealing ring 42 contacted or engaged with the plunger 30 for making an air or water or fluid tight seal between the insert 40 and the plunger 30.

A packing 50 is disposed or engaged into the compartment 32 at the other end portion 34 of the plunger 30 and releasably attached or mounted or secured or anchored or retained to the plunger 30 with a force-fitted engagement, for forming or defining or creating a vacuum 80 within the plunger 30 and between the insert 40 and the packing 50, and an extension or actuator or engaging member 51 is attached or mounted or secured to the packing 50 and partially extended out of the plunger 30 (FIG. 3) for selectively engaging with the needle seat 21 (FIGS. 1, 4-6), and for selectively drawing or moving or pulling the needle 20 into the compartment 32 or the vacuum 80 of the plunger 30 after use (FIG. 7).

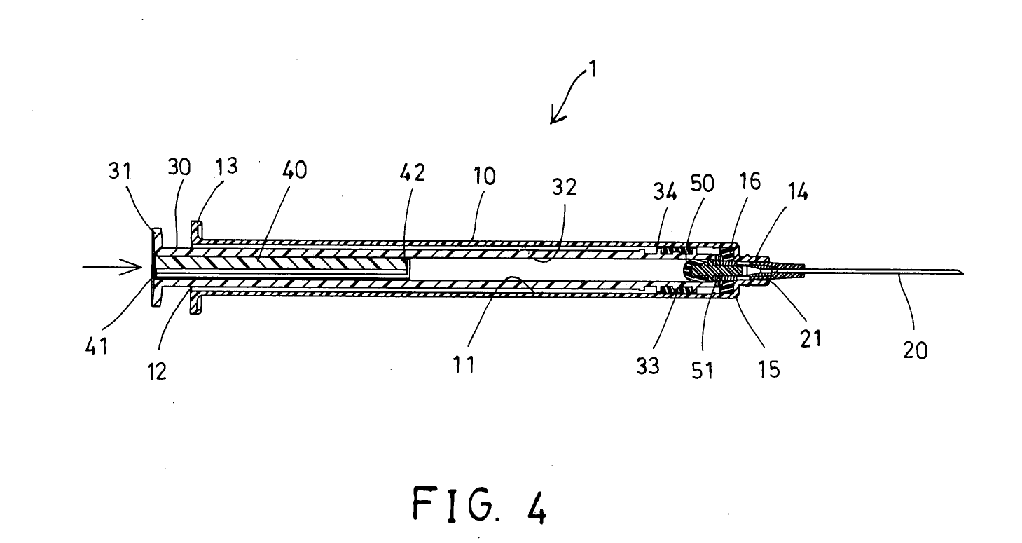

In operation, as shown in FIG. 1, the vacuum 80 is formed or provided in the compartment 32 of the plunger 30 and between the insert 40 and the packing 50, and the packing 50 is releasably attached or mounted or secured or anchored or retained to the barrel 10 before the plunger 30 is slidably received or engaged in the chamber 11 of the barrel 10. As shown in FIG. 3, when the plunger 30 is drawn or moved or pulled out of the barrel 10 or away from the needle 20, the fluid or medicine may be selectively drawn into the chamber 11 of the barrel 10, and may then be selectively forced to move or to flow out through the needle 20 (FIG. 4) for injection purposes by moving or pressing the plunger 30 into the barrel 10.

As shown in FIG. 5, after the fluid or medicine injection operation, the plunger 30 may further be moved or pressed into the barrel 10 and toward the needle seat 21 and the needle 20, to have or to cause or to force the engaging member 51 to engage with the needle seat 21, such as to engage with the cone-shaped or frustum-shaped or tilted or inclined inner peripheral surface 22 of the needle seat 21 for solidly and stably attaching or mounting or securing the engaging member 51 to the needle seat 21.

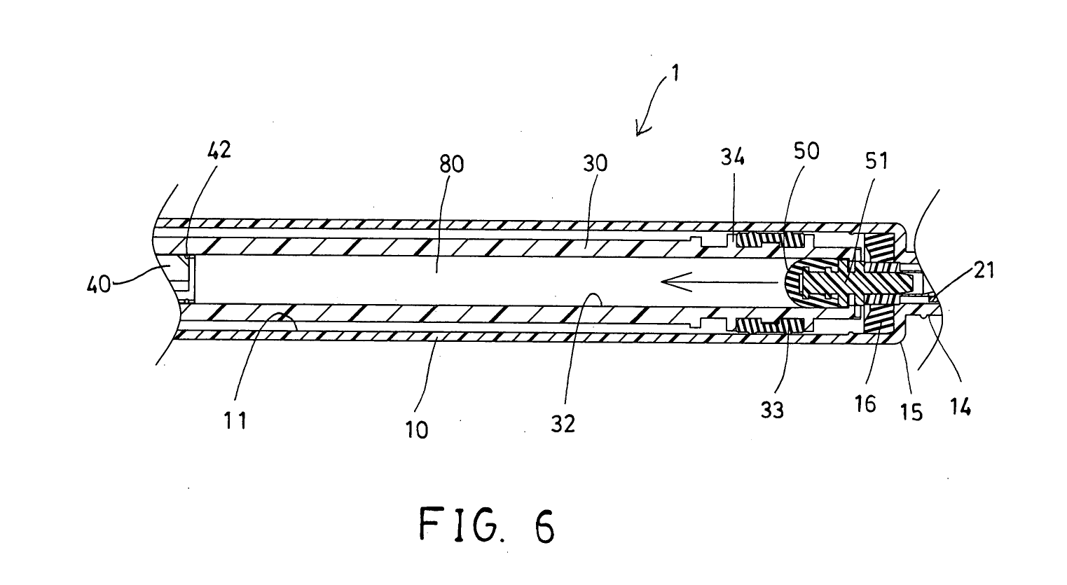

Simultaneously, when the other end portion 34 of the plunger 30 is forced or moved or pressed onto the soft or resilient gasket 16, and when the engaging member 51 is further forced or moved or pressed to engage with the needle seat 21 that is engaged in the mouth 14 of the barrel 10 (FIG. 6), due to a reaction force, the engaging member 51 and the packing 50 may be forced to be moved relative to the plunger 30, and a releasable engagement between the plunger 30 and the packing 50 may be spoiled, and the packing 50 and the engaging member 51 and the needle seat 21 and the needle 20 may then be forced to move or may be automatically drawn into the compartment 32 or the vacuum 80 of the plunger 30 by the vacuum 80 of the plunger 30 (FIG. 7).

It is to be noted that the automatic retractable safety syringe in accordance with the present invention includes a simplified structure having the insert 40 and the packing 50 engaged into the compartment 32 of the plunger 30 for forming or defining or creating a vacuum 80 within the plunger 30, and the plunger 30 may then be easily and quickly disposed or engaged or assembled into the chamber 11 of the barrel 10, and the automatic retractable safety syringe may be easily used or operated as that of the typical syringes.

Accordingly, the automatic retractable safety syringe in accordance with the present invention includes a simplified structure that may be easily and quickly manufactured and assembled and that may include a greatly reduced or decreased manufacturing cost, and that may be easily actuated or operated by the user.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A syringe comprising:
a barrel including a chamber formed therein, and including an opening formed in a first end portion thereof, and including a mouth formed in a second end portion thereof,
a needle seat engaged in said mouth of said barrel and including a needle attached to said needle seat,
a plunger slidably engaged into said opening and said chamber of said barrel, and including a first end portion located out of said barrel, and including a compartment formed therein, and including a second end portion, and
a packing releasably engaged into said compartment at said second end portion of said plunger for forming a vacuum within said plunger, and an engaging member attached to said packing and extended out of said plunger for selectively engaging with said needle seat and for selectively moving said needle seat and said needle into said compartment of said plunger, and
said packing and said engaging member being forced to move relative to said plunger and to spoil a releasable engagement between said plunger and said packing when said plunger is forced and moved toward said needle seat, and said packing and said engaging member and said needle seat and said needle being automatically drawn into said compartment of said plunger by said vacuum of said plunger when said packing is forced to move relative to said plunger.

2. The syringe as claimed in claim 1, wherein said barrel includes a gasket engaged into said chamber of said barrel and located close to said mouth of said barrel for engaging with said second end portion of said plunger.

3. The syringe as claimed in claim 1, wherein said plunger includes an insert engaged into said compartment at said first end portion of said plunger for forming said vacuum within said plunger and between said insert and said packing.

4. The syringe as claimed in claim 3, wherein said insert is secured to said barrel with a latch device.

5. The syringe as claimed in claim 3, wherein said insert includes a sealing ring engaged with said plunger for making a fluid tight seal between said insert and said plunger.

6. The syringe as claimed in claim 1, wherein said plunger includes a sealing ring disposed on said second end portion of said plunger and engaged with said barrel for making a fluid tight seal between said barrel and said plunger.
